SEQUENCE LISTING (1 sequence)

SEQ ID NO: 1 - DNA, 6912 bp, Phage M13mp18
  bacteriophage DNA
ttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttg

(19) United States
(12) Patent Application Publication
(10) Pub. No.: US 2023/0227892 A1
(43) Pub. Date: Jul. 20, 2023

(54) Method of Determining a Quantitative Fingerprint of a Subset of Bacteria in a Person's Gastrointestinal Microbiome

(71) Applicant: mbiomics GmbH, Munich (DE)

(72) Inventors: Johannes Benedikt Woehrstein, Hohenbachern (DE); Heinrich Ernst Paul Grabmayr, Munich (DE)

(21) Appl. No.: 17/576,865

(22) Filed: Jan. 14, 2022

(51) Int. Cl. C12Q 1/6816; C12Q 1/6876

(52) U.S. Cl. CPC C12Q 1/6816; C12Q 1/6876

(57) ABSTRACT

The relative abundance of bacterial species in a patient’s microbiome is quantified using DNA nanostructures that fluoresce multiple colors. Immobilizing binders have binding sites with nucleotide sequences complementary to those at a primary site on rRNA subunits of each selected bacterial species. Fluorophore binders have binding sites with nucleotide sequences complementary to those at a secondary site on the rRNA subunits. The fluorophore binders for each bacterial species are attached to nanostructures that fluoresce a particular color for each bacteria. The immobilizing binders are attached to the surface of a microscopy chamber. RNA subunits are extracted from a microbiome sample of the patient and are attached to the corresponding immobilizing binders and fluorophore binders such that the RNA subunits of each bacterial species fluoresce a color unique to the species. DNA nanostructures emitting the same color are counted to determine the relative concentration of the bacterial species in the sample.

### REFERENCE TO SEQUENCE LISTING SUBMITTED ELECTRONICALLY

The content of the electronically submitted sequence listing (Name: MBM-001_Seqlisting.txt: Size: 9,333 bytes; and Date of Creation: Jan. 14, 2022) is incorporated herein by reference in its entirety.

### TECHNICAL FIELD

The present invention relates to the field of microbiome analysis and particularly to a method for determining a quantitative fingerprint of a subset of bacteria in a person’s gastrointestinal microbiome.

### BACKGROUND INFORMATION

Microbiomes inside the human gastrointestinal tract play an important role in the immune system, metabolism and general health. The identity and abundance of the various species (also referred to herein as strains) of bacteria in the human microbiome can be correlated to a number of conditions and diseases, such as cardiovascular disease, hypertension, cancer, diabetes, obesity and auto-immune diseases. In addition, the composition of bacteria in the gastrointestinal microbiome may also be a proxy for the probability of adverse reactions to medical treatments. Thus, various conditions and diseases can be diagnosed and various therapies prescribed by identifying and quantifying the relative presence of specific bacteria in a person’s microbiome. One way of identifying and qualifying bacteria relies on the 16S component of ribosomal RNA. The 16S rRNA genes of most bacteria in the human gastrointestinal tract have been sequenced and are listed in publicly accessible databases, such as the GenBank of the National Institute of Health. One method of identifying the various bacteria in a patient’s fecal (stool) sample involves analyzing the 16S rRNA of the bacteria using quantitative polymerase chain reaction (qPCR) analysis. However, qPCR amplification can be time consuming, inaccurate and a poor measure of the relative abundance of the various bacteria that are identified.

Another method of identifying the various bacteria strains involves shotgun metagenomic sequencing and is based on sequencing the total extracted DNA of the gastrointestinal microbiome, which is bioinformatically decomposed into the known genomes of the potential bacteria. The benefits compared to 16S analysis are stain-level identification, high sensitivity, and the possibility to use the genetic information for further analysis. However, because of the highly complex reconstruction of the microbiome composition, shotgun metagenomic sequencing provides poor quantification.

One problem with current microbiome analysis methods is the limited range of the various concentrations that can be measured. The concentrations of the various types of bacteria in the human gastrointestinal tract can vary by orders of magnitude. The accuracy of current methods for quantifying multiple bacteria strains in a single sample is lower when the concentrations of the bacteria strains vary by orders of magnitude. For example, Bacteroides vulgatus, Bacteroides uniformis, and Alistipes putredinis are often present in human stool samples at a population size of 1-10%, while Bacteroides fragilis and Bacteroides coprophilus are present at a population size of 0.01%-0.1%, and Abiotrophia defective and Acidaminococcus fermentans occur at a population size of 0.00001%-0.0001%. Current analysis methods cannot accurately quantify the relative concentrations of these bacteria strains in a single sample.

A method is sought that allows multiple target strains of bacteria in a gastrointestinal microbiomic sample to be accurately quantified over a wide range of concentrations of the target species and in a shorter analysis time than required for existing analysis methods.

### SUMMARY

A method for determining a quantitative fingerprint of a predetermined subset of bacterial species in a gastrointestinal tract sample of a patient uses small DNA nanostructures to detect and count single nucleic acid molecules of the various target bacterial species. The method involves attaching a DNA nanostructure that fluoresces a predetermined fluorophore color to a ribosomal RNA (rRNA) subunit of the target bacterial species that corresponds to the predetermined color. The relative concentrations of the various bacterial species in the predetermined subset are determined based on how many DNA nanostructures with the predetermined fluorophore color for each target bacterial species are counted on the surface of a microscopy chamber. The quantitative fingerprint of bacterial abundance in the patient’s gastrointestinal tract is compared with the known relative abundance indicative of a particular disease or condition. A therapy is administered in order to decrease the relative concentration of a particular strain of bacteria indicated by the quantitative fingerprint to be in overabundance in a patient’s gastrointestinal tract compared to the bacterial concentrations in a healthy person.

A first bacterial species of the predetermined subset is selected. A primary binding site and a secondary binding site on a first rRNA subunit of the first bacterial species are selected. First immobilizing binders are formed that include a nucleotide sequence complementary to that of the primary binding site on the first rRNA subunit. First fluorophore binders are formed that include a nucleotide sequence complementary to that of the secondary binding site on the first rRNA subunit. The first fluorophore binders are attached to first DNA nanostructures that each includes parallel DNA double helices forming a flat shape. Each first DNA nanostructure has a maximum length of less than 150 nm and is attached to a first organic fluorophore with a first color, a second organic fluorophore with a second color, and a third organic fluorophore with a third color. The first DNA nanostructures exhibit a first fluorophore color produced by a first combination of intensities of the first color, the second color and the third color.

A second bacterial species of the predetermined subset is selected. A primary binding site and a secondary binding site on a second rRNA subunit of the second bacterial species are selected. Second immobilizing binders are formed that include a nucleotide sequence complementary to that of the primary binding site on the second rRNA subunit. Second fluorophore binders are formed that include a nucleotide sequence complementary to that of the secondary binding site on the second rRNA subunit. The second fluorophore binders are attached to second DNA nanostructures that each includes parallel DNA double helices. Each second DNA nanostructure has a maximum length of less than 150 nm and is attached to a fourth organic fluorophore with the first color, a fifth organic fluorophore with the second color, and a sixth organic fluorophore with the third color. The second DNA nanostructures exhibit a second fluorophore color produced by a second combination of intensities of the first color, the second color and the third color.

Immobilizing oligonucleotides are attached to the surface of the microscopy chamber. The first immobilizing binders are attached to a first group of the immobilizing oligonucleotides, and the second immobilizing binders are attached to a second group of the immobilizing oligonucleotides. Ribosomal RNA subunits are extracted from bacteria present in the gastrointestinal microbiomic sample from the patient. The extracted rRNA subunits are added to the microscopy chamber. Hybridization reactions are performed to bind the first immobilizing binders to the primary binding site on first rRNA subunits present in the microscopy chamber, and to bind the second immobilizing binders to the primary binding site on second rRNA subunits present in the microscopy chamber. Hybridization reactions are performed to bind the first fluorophore binders to the secondary binding site on first rRNA subunits present in the microscopy chamber, and to bind the second fluorophore binders to the secondary binding site on second rRNA subunits present in the microscopy chamber.

Image analysis is performed to detect the first fluorophore color and thereby to count each first DNA nanostructure that is immobilized on the surface of the microscopy chamber. Image analysis is also performed to detect the second fluorophore color and thereby to count each second DNA nanostructure that is immobilized on the surface of the microscopy chamber. The relative concentration of the first bacterial species compared to the second bacterial species is determined based on how many first DNA nanostructures and how many second DNA nanostructures are counted on the surface of the microscopy chamber.

In another implementation, the optimal relative concentration of the first bacterial species compared to the second bacterial species is determined in order to improve a medical condition of the patient from whom the gastrointestinal microbiomic sample was taken. Then a dietary supplement is administered to the patient that changes the relative concentration of the first bacterial species compared to the second bacterial species so as to move the patient’s microbiome closer to the optimal relative concentration.

Further details and embodiments and methods are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 is a table of selected species of bacteria as well as their relative abundance in a healthy person.

FIGS. 2A and 2B show a flowchart of steps of a novel method for quantifying the abundance of various species of bacteria in a patient’s gastrointestinal tract.

FIG. 3 is a schematic diagram of a 16S ribosomal RNA subunit of a bacteria attached to an immobilizing binder and a fluorescent DNA nanostructure.

FIG. 4 is a perspective schematic diagram of an exemplary DNA nanostructure of the type used in the method of FIGS. 2A and 2B for quantifying bacteria.

FIG. 5 is a top view of the DNA nanostructure shown in FIG. 4.

FIG. 6 shows more detail of how organic fluorophores are attached to the DNA nanostructure of FIG. 4.

FIGS. 7A, 7B, 7C and 7D show the layout and nucleotide sequences of the DNA nanostructure of FIG. 4 without the attached fluorophores.

FIGS. 8A, 8B, 8C and 8D show the nucleotide sequence (SEQ ID No: 1) of a circular DNA strand used to make the DNA nanostructure of FIG. 4.

FIG. 9 is a schematic diagram of a second embodiment by which a 16S ribosomal RNA subunit of a bacteria is attached to an immobilizing binder and thereby immobilized on the surface of a microscopy chamber.

FIG. 10 shows a digital image generated using the method of FIGS. 2A and 2B which includes multi-colored spots corresponding to particular bacterial species.

FIG. 11 is a schematic diagram of another embodiment by which a 16S ribosomal RNA subunit of a bacteria is attached to two separate fluorophore binders and thereby two fluorescent DNA nanostructures.

FIG. 12 is a schematic diagram of another embodiment by which a 16S ribosomal RNA subunit of a bacteria is attached to three separate fluorophore binders and thereby three single-color fluorescent DNA nanostructures.

### DETAILED DESCRIPTION

Reference will now be made in detail to some embodiments of the invention, examples of which are illustrated in the accompanying drawings.

The novel method for determining a quantitative fingerprint of a predetermined subset of bacterial species in a gastrointestinal microbiomic sample uses a multiplexed nucleic acid detection assay to detect and count single target nucleic acid molecules of the various bacterial species. Fluorescent nucleic acid nanostructures (also called deoxyribonucleic acid (DNA)-based nanostructures) are used to detect and quantify 16S rRNA molecules of the bacteria at the single molecule level. The target nucleic acid molecules of the bacteria are immobilized on a surface. A fluorescent DNA nanostructure is then hybridized with an immobilized target nucleic acid molecule. Distinct target nucleic acid sequences of the various bacteria are labeled with a particular fluorescent DNA nanostructure having a distinct color that can be distinguished using fluorescence microscopy. Each distinct target nucleic acid molecule of a bacteria is quantified by imaging the fluorescent DNA nanostructures using the fluorescence microscopy. The individual fluorescent DNA nanostructures that are attached to the target nucleic acid molecules are identified and counted. In this manner, the relative number of single target nucleic acid molecules can be simultaneously and quickly analyzed.

The relative number of individual bacterial molecules forms a quantitative fingerprint of predetermined bacterial strains that is used for prognostic and therapeutic purposes. For example, a particular subset of bacteria present in a patient’s gastrointestinal tract at specific relative concentrations can be correlated to a number of diseases, such as Parkinson’s disease, Alzheimer’s disease, irritable bowel disease, atherosclerotic cardiovascular disease, late stage melanoma and colon cancer. An entire field of microbiome diagnostics has emerged. For example, of the hundreds of bacterial species that are likely present, a limited subset (such as 5 to 124) of strains can be chosen for which the relative abundance for a normal healthy patient is known or for which the relative abundance indicative of a particular disease or condition is known. The quantitative fingerprint of bacterial abundance of the patient obtained using the novel method is then compared with the known relative abundance indicative of the particular disease or condition. Moreover, a particular quantitative fingerprint of another subset of species of bacteria can indicate that the patient will likely have a favorable response to an immunotherapy for cancer, such as late stage melanoma.

In another example, a therapy can be administered in order to decrease the relative concentration of a particular strain of bacteria indicated by the quantitative fingerprint to be in overabundance in a patient’s gastrointestinal tract compared to the bacterial concentrations in a healthy person. Alternatively, the patient can be directed to consume probiotics (live bacteria) or prebiotics (carbohydrates beneficial to bacteria) in order to increase the relative concentration of a particular strain of bacteria indicated by the quantitative fingerprint to be insufficiently present in the patient’s gastrointestinal tract. The relative concentrations of particular species of bacteria can also be increased or decreased in order to come closer to the average historical quantitative bacterial distribution for a specific patient as opposed to the bacterial concentrations of a typical healthy person. This alternative requires a quantitative fingerprint of bacterial species in the patient’s gastrointestinal tract to be periodically determined and stored in order to determine the average historical quantitative fingerprint.

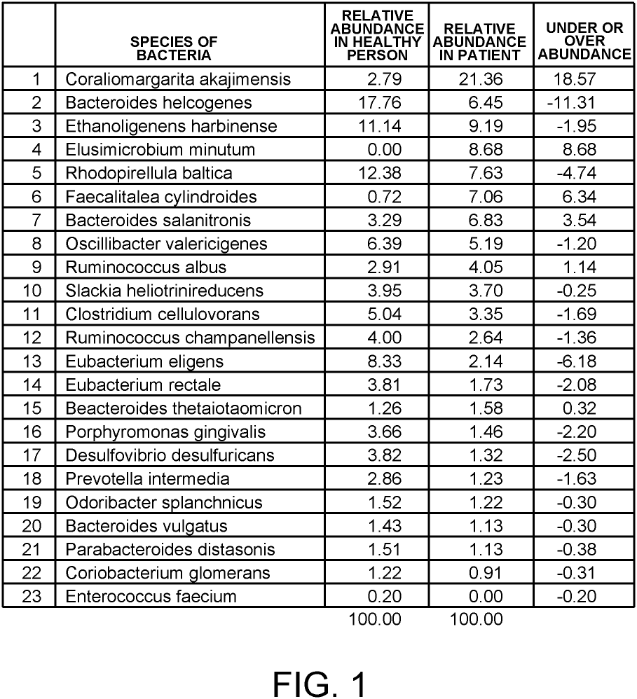

In yet another example, the novel method is used to generate a quantitative fingerprint of twenty-three predetermined bacterial strains. FIG. 1. lists the predetermined bacterial species as well as their relative abundance (normalized for the 23 species) in a healthy person. The method is used to determine the relative abundance of each of the twenty-three species of bacteria in a patient’s gastrointestinal tract. For example, the method determines that there is a large overabundance of the first listed bacteria Coraliomargarita akajimensis and a large underabundance of the second listed bacteria Bacteroides helcogenes. The method is now described for the simplified case of determining the relative concentrations of two species of bacteria, such as just the first two species listed in FIG. 1.

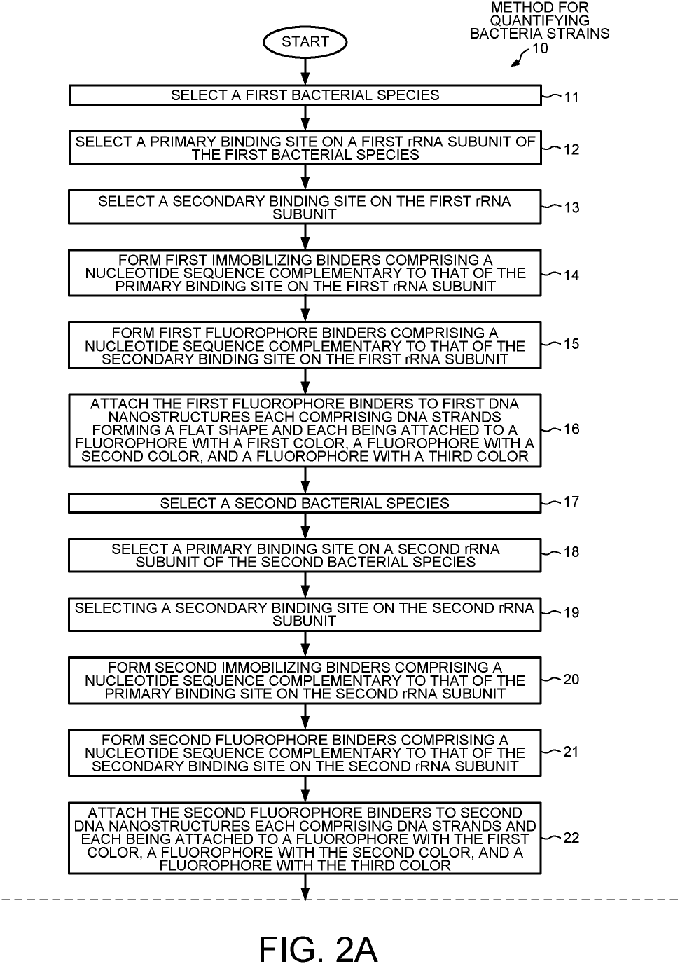

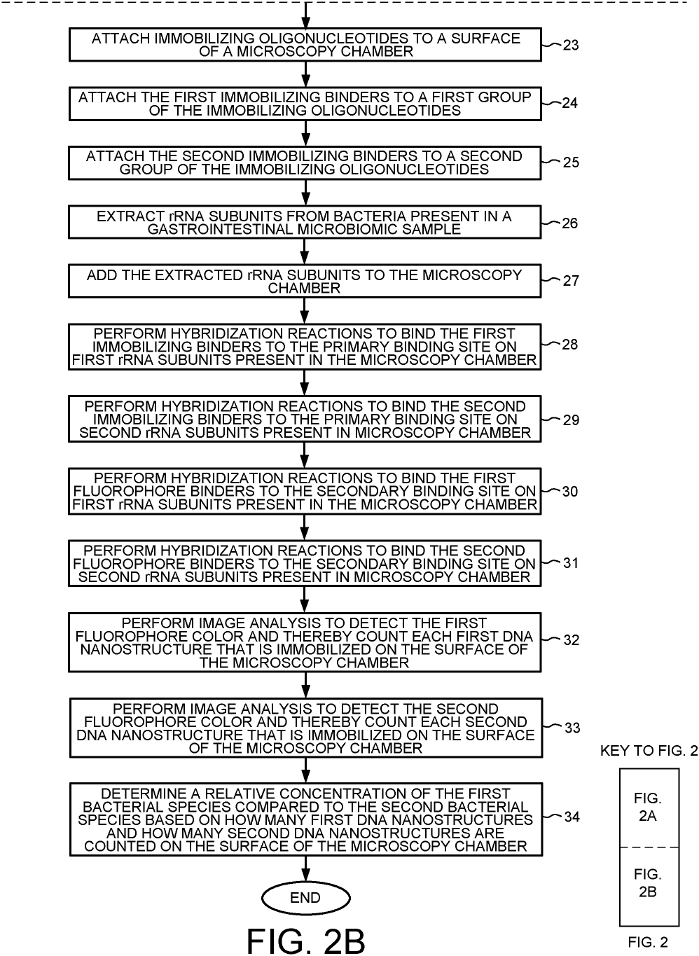

FIGS. 2A and 2B show a flowchart of steps 11-34 of the novel method 10 for determining a quantitative fingerprint of a subset of bacteria in a patient’s gastrointestinal microbiome. The method steps are described for quantifying two species of bacteria.

In a first step 11, a subset is chosen of the various bacterial species likely to be present in the patient’s gastrointestinal tract, including a first bacterial species such as Coraliomargarita akajimensis.

In step 12, a primary binding site is selected on a first ribosomal RNA (rRNA) subunit of the first bacterial species. Thus, the first rRNA subunit corresponds to the first bacteria Coraliomargarita akajimensis, and a second rRNA subunit corresponds to the second bacteria Bacteroides helcogenes. Each rRNA subunit is chosen to have more than 1000 nucleotides and less than 5000 nucleotides. The rRNA subunit can be a 16S ribosomal RNA subunit, an 18S ribosomal RNA subunit, a 23S ribosomal RNA subunit, or a 28S ribosomal RNA subunit. In one embodiment, the rRNA subunit is a 16S ribosomal RNA subunit. The 16S subunit of the bacterial ribosome has a Svedberg sedimentation coefficient of 16S. The 16S subunit of ribosomal RNA is used to identify and quantify bacteria. The 16S rRNA genes of most bacteria in the human gastrointestinal tract have been sequenced and are listed in publicly accessible databases, such as the GenBank of the National Institute of Health.

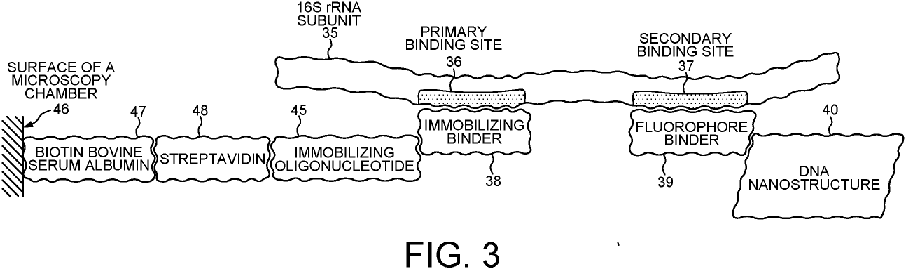

FIG. 3 illustrates the first 16S rRNA subunit 35, which is to be counted using method 10. However, at step 12, the first 16S rRNA subunit 35 has not yet been purified from a patient sample. Subunit 35 is shown here to illustrate the nucleotide sequences that are selected as binding sites on the subunit 35. The primary binding site 36 on the first rRNA subunit 35 is selected so as to include more than eighteen and less than twenty-six nucleotides and to constitute a unique sequence from among all of the 16S rRNA sequences of the various bacteria listed in the database. The primary binding site 36 is also selected so as to include no more than four repetitive nucleotides and to include nucleotides comprising 35%-55% Guanine and Cytosine. In one embodiment, the primary binding site 36 on the first rRNA subunit 35 is a unique sequence of twenty nucleotides. In another embodiment, the primary binding site on the first rRNA subunit includes at least twenty nucleotides separated into a first sequence having at least ten nucleotides and a second sequence having at least ten nucleotides separated by a region of nucleotides that do not belong to the primary binding site.

In step 13, a secondary binding site 37 on the first rRNA subunit 35 is selected in a manner analogous to that used to select the primary binding site 36.

In step 14, first immobilizing binders 38 are formed that include a nucleotide sequence complementary to that of the primary binding site 36 on the first rRNA subunit 35.

In step 15, first fluorophore binders 39 are formed that include a nucleotide sequence complementary to that of the secondary binding site 37 on the first rRNA subunit 35.

In step 16, the first fluorophore binders 39 are attached to first DNA nanostructures 40 that each include parallel DNA double helices that form a flat shape.

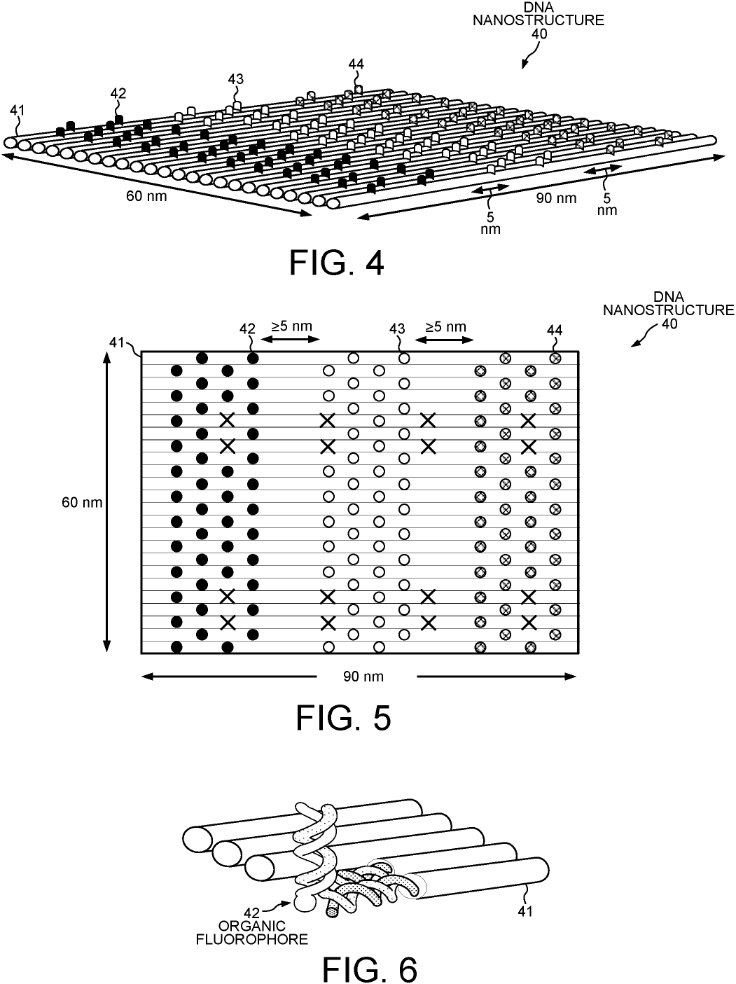

FIG. 4 is a perspective schematic diagram of an exemplary DNA nanostructure of the type used in method 10 for quantifying bacteria, such as first DNA nanostructure 40. The DNA nanostructures used in method 10 are made by folding double-helix DNA strands into a flat structure, which is only slightly curved and has a rippled surface. Various shapes of the flat structure can be made, such as rectangular, circular or triangular. The DNA nanostructures have maximum lengths of less than 110 nm. First DNA nanostructure 40 is rectangular with dimensions of 60 nm by 90 nm. Longer DNA rail strands are held in place by shorter rung strands that connect locations on the longer folded rail strands.

FIG. 4 shows that first DNA nanostructure 40 is formed from twenty-four, parallel DNA rail strands, such as rail strand 41 that forms the back side of the rectangle. The DNA rail strands are held in the rectangular shape by 184 rung strands. FIG. 5 is a top view of first DNA nanostructure 40.

Many fluorescence dye molecules (called organic fluorophores) are attached to each DNA nanostructure. The organic fluorophores can be fluorescent proteins (such as RFP, GFP or YFP), an Atto® dye (such as Atto647N, Atto565 or Atto488), a cyanine dye (such as Cy3), an Alexa® dye, a rhodamine dye, a coumarin dye, or the blue-fluorescent 4′,6-diamidino-2-phenylindole (DAPI). The organic fluorophores are attached to the DNA rail strands using connectors formed by twenty-one nucleotides that attach to the 3′-end of the DNA strand. A complementary connector attaches to the DNA nanostructure. FIGS. 4-5 illustrate where the first DNA nanostructure 40 is attached to a first organic fluorophore 42 with a first color, a second organic fluorophore 43 with a second color, and a third organic fluorophore 44 with a third color. Each of the exemplary organic fluorphores 42-44 is attached to the DNA rail strand 41. FIG. 6 shows more detail of how first organic fluorophore 42 is attached to the double-helix DNA strand of rail strand 41.

In one implementation, the first color is red, and first organic fluorophore 42 is Atto 647N. The second color is green, and second organic fluorophore 43 is Cy3. The third color is blue, and third organic fluorophore 44 is Atto 488. In order for the fluorescence microscopy system better to distinguish the wavelengths from the various fluorescence colors, fluorophores are chosen that fluoresce at wavelengths that differ by at least 25 nm. For example, the three organic fluorophores on DNA nanostructure 40 are chosen such that the second organic fluorophore 43 has a wavelength of maximum absorption that is at least 25 nm larger than the wavelength of maximum emission of the third organic fluorophore 44, and the first organic fluorophore 42 has a wavelength of maximum absorption that is at least 25 nm larger than the wavelength of maximum emission of the second organic fluorophore 43. In the embodiment shown in FIGS. 4-5, there are forty-four fluorophore molecules of each color disposed within the rectangular DNA nanostructure 40 of 60 nm × 90 nm. Because the size of the DNA nanostructure is so small, the variously colored light emitted by different fluorophores combines and is perceived by a fluorescence microscopy system as a single multi-colored spot. Thus, the first DNA nanostructures exhibit a first fluorophore color produced by a first combination of intensities of the first color, the second color and the third color.

Moreover, the relative intensities of the differently colored light contribute to the single combined color emitted from each DNA nanostructure. Although theoretically an arbitrarily large number of emitted colors can be generated by varying the intensities of light from the differently colored fluorophores, the plurality of emitted colors can best be distinguished from one another if the intensity of the colored light from the different fluorophores is changed in discrete levels. The number of possibilities for the single combined color emitted from each DNA nanostructure is N=L^C, where L is the number of intensity levels of each fluorophore color, and C is the number of fluorophore colors. For example, with two fluorophore colors and three intensity levels of those colors, nine single combined colors are possible. With three fluorophore colors and two intensity levels of those colors, eight single combined colors are possible. The first intensity level could be achieved by attaching forty-four fluorophore molecules to the DNA nanostructure, and the second intensity level could be achieved by attaching only twenty-two fluorophore molecules to the DNA nanostructure.

With three fluorophore colors and four intensity levels of those colors, sixty-four single combined colors are possible. And with three fluorophore colors and five intensity levels of those colors, 125 single combined colors are possible. Where one of the intensity levels is chosen to be zero intensity (or black), then no combined color is generated where the intensity level of all colors is zero because that color is black, which is indistinguishable from the non-emitting portions of the DNA nanostructure or any location on the surface of the microscopy chamber where no DNA nanostructures are present. Thus, if there are four intensities plus zero intensity for each of three colors, then there are 124 possible combined fluorophore colors. In the example shown in FIGS. 4-5, the three differently colored fluorophores have the same number of fluorophore molecules and, therefore, emit the same intensity of each of the three colors. The resulting combined color is just one of the 124 possible combined colors that can be generated with three original colors and four intensities plus black (zero intensity). Using this intensity and color implementation, a panel of 124 bacteria can be quantified using the small DNA nanostructures all having the same basic configuration. The small form factor of the DNA nanostructures is advantageous because the nanostructures attached to each rRNA subunit do not inhibit the rRNA subunits from attaching and being immobilized in a compact manner on the imaging surface of a microscopy chamber.

Despite the advantage of being able to generate a very large number of colors on a very small DNA nanostructure, the organic fluorophore molecules of different colors must still be spaced apart from each other by a minimum distance in order to prevent Foerster resonance energy transfer (FRET), a non-radiative process whereby energy (and thereby fluorescent intensity) from an excited fluorophore is lost to a nearby fluorophore. Energy is transferred from a fluorophore with a higher energy state to a nearby fluorophore with a lower energy state. Because shorter wavelengths are associated with higher energy, energy is lost through FRET transfer to fluorophores with colors associated with longer wavelengths. For example, if there are three nearby fluorophores with the colors red, green, and blue, then FRET energy transfer occurs from the blue fluorophore to the green fluorophore and from the blue and green fluorophores to the red fluorophore. However, the red fluorophore does not lose energy (or fluorescent intensity) because there is no nearby fluorophore with an even lower energy state to which its energy can be transferred. Moreover, FRET transfer does not occur between fluorophores of the same color.

If the intensity of a fluorophore of one color is lost to a nearby fluorophore of another color, then the single combined color of the DNA nanostructure would change and would be difficult to control. For purposes of the organic fluorophores used to make DNA nanostructure 40, FRET transfer is significant only within a FRET distance of about 5 nm. Thus, the organic fluorophore molecules can best be separated by color on the flat rectangular DNA nanostructure 40 by grouping common fluorophores in stripes and separating the stripes by at least 5 nm. For example, where fluorophores are used that have three distinct colors, the fluorophores are attached to the rectangular DNA nanostructure in three stripes separated by areas without fluorophores between the stripes, as illustrated in FIGS. 4-5. Thus, no first organic fluorophore 42 is attached to any of the first DNA nanostructures at a location within 5 nm of any second organic fluorophore 43 or third organic fluorophore 44. Then by varying the number of fluorophores attached to the DNA nanostructure 40 within each stripe (i.e., the density of attached fluorophores), the intensity of the fluorescence emitted from each stripe can be controlled. By controlling the intensities of the component colors emitted from the variously colored fluorophores to discrete levels, the various single combined colors emitted from the DNA nanostructures can best be distinguished from one another.

In the embodiment shown in FIGS. 4-5, there are forty-eight positions for fluorophore molecules in the matrix forming the stripe for each color. However, four of the forty-eight positions are not occupied by fluorophore molecules because those positions are used to attach binding oligonucleotides that connect the DNA nanostructure 40 to the associated fluorophore binder 39. Two matrix positions are used to attach each binding oligonucleotide. The fluorophore binders 39 include a nucleotide sequence complementary to that of the binding oligonucleotides that protrude from the surface of the DNA nanostructure 40. In FIG. 5, there are eight pairs of attachment positions marked by Xs at which the first fluorophore binders 39 are attached to the first DNA nanostructures 40 in step 16. In another embodiment, ten pairs of attachment positions are used to attach the fluorophore binders to the DNA nanostructures.

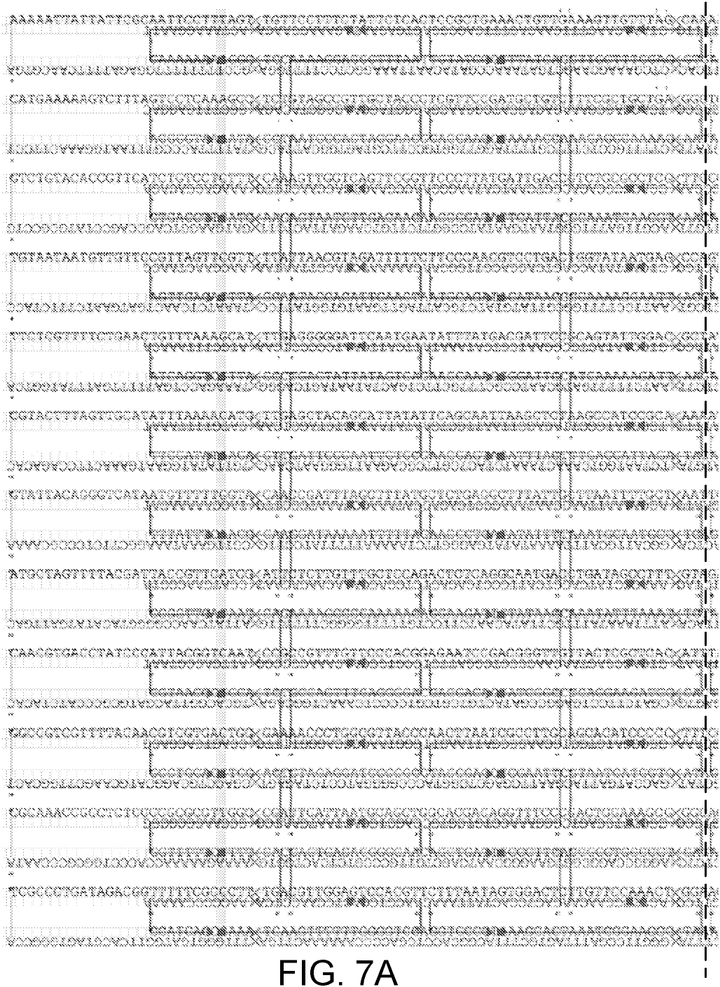

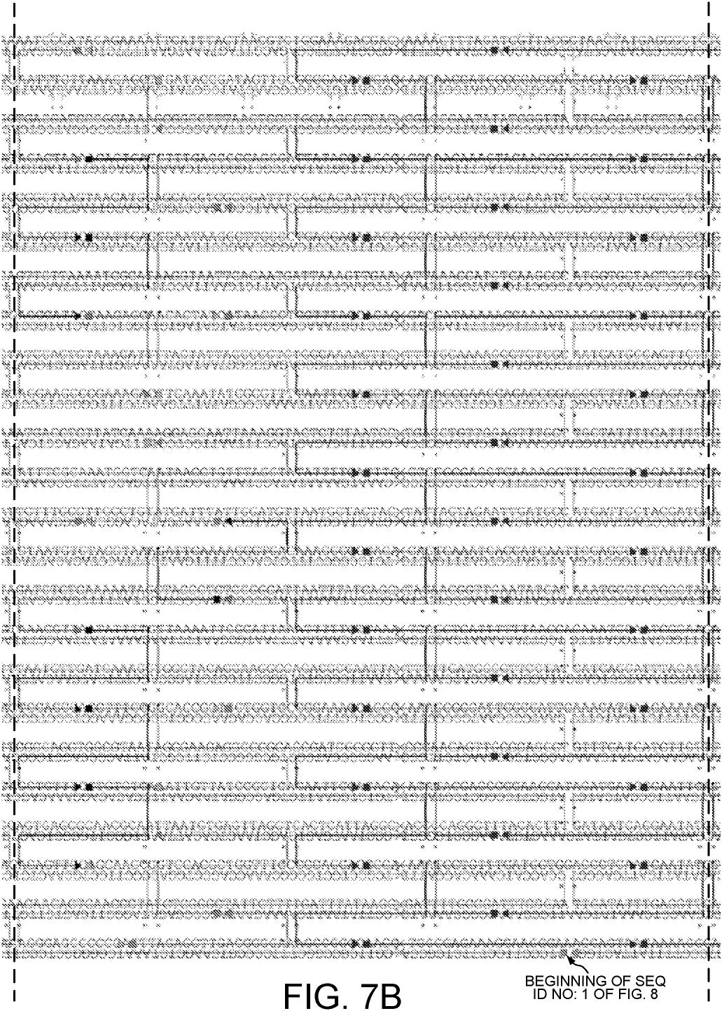

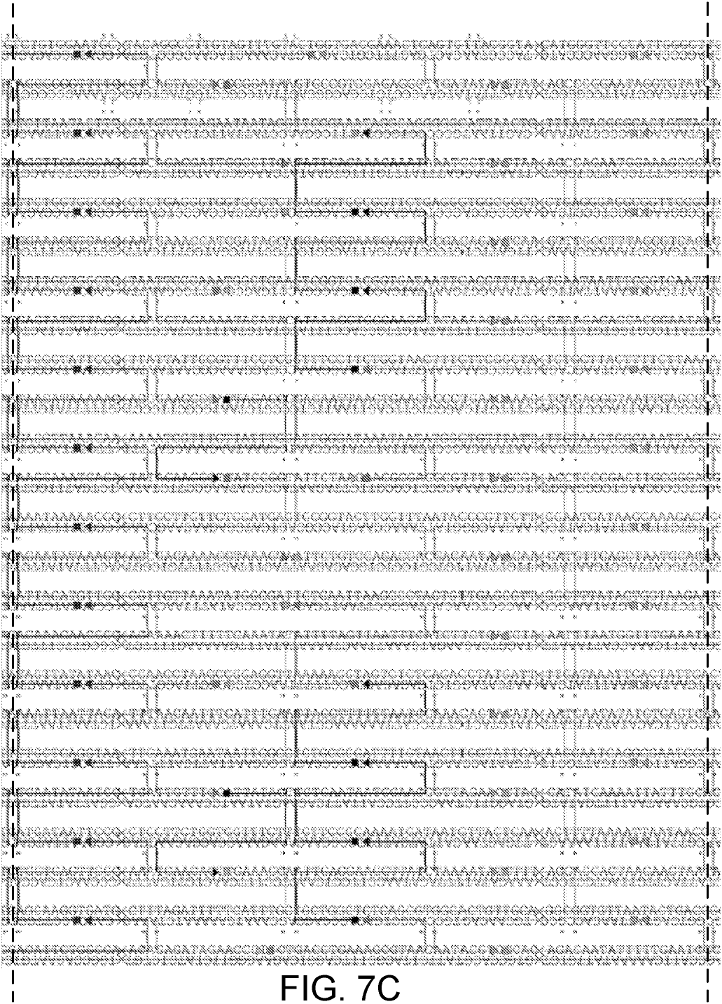

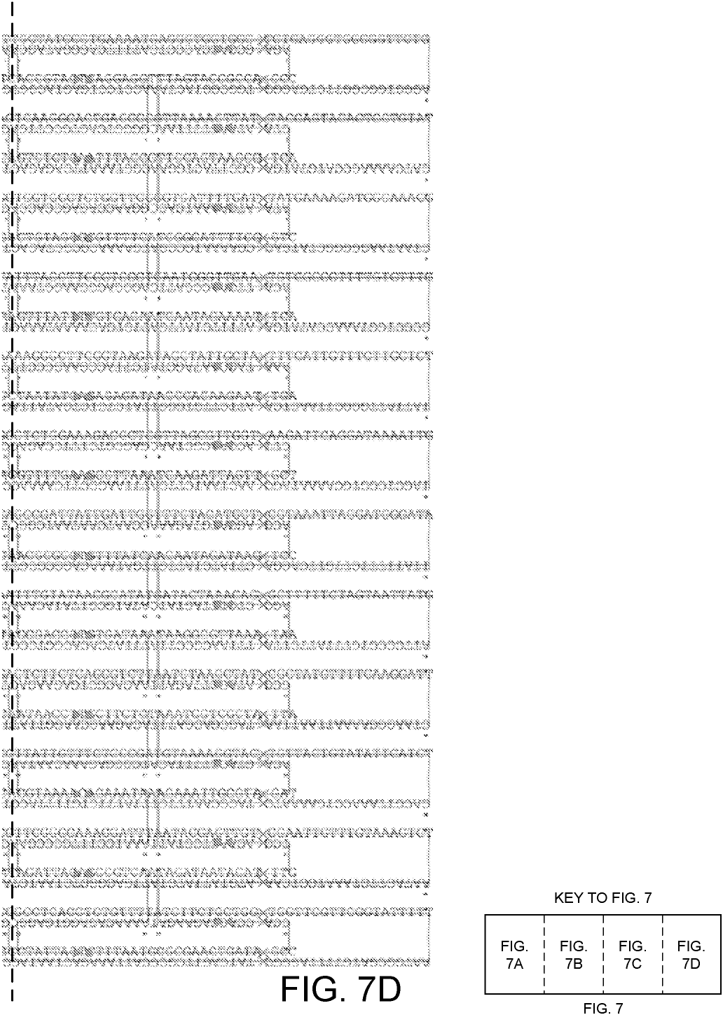

FIGS. 7A, 7B, 7C and 7D show the layout of DNA nanostructure 40 and the associated nucleotide sequences as generated by the caDNAno design tool. The twenty-four, parallel DNA rail strands are formed by routing the single, circular DNA scaffold strand into loops. Rung strands then connect parts of the scaffold to form the rectangle. Eight rung strands are biotinylated on the 5′ end. Most of the 3′ and 5′ ends of the rung strands are disposed on the same face of the rectangular DNA nanostructure 40. In addition, the organic fluorophores protrude from the same face of the nanostructure. The Xs in the sequences of the nucleotide layout of FIGS. 7A, 7B, 7C and 7D indicate that a nucleotide was skipped, which is required in order to prevent the DNA nanostructure from twisting.

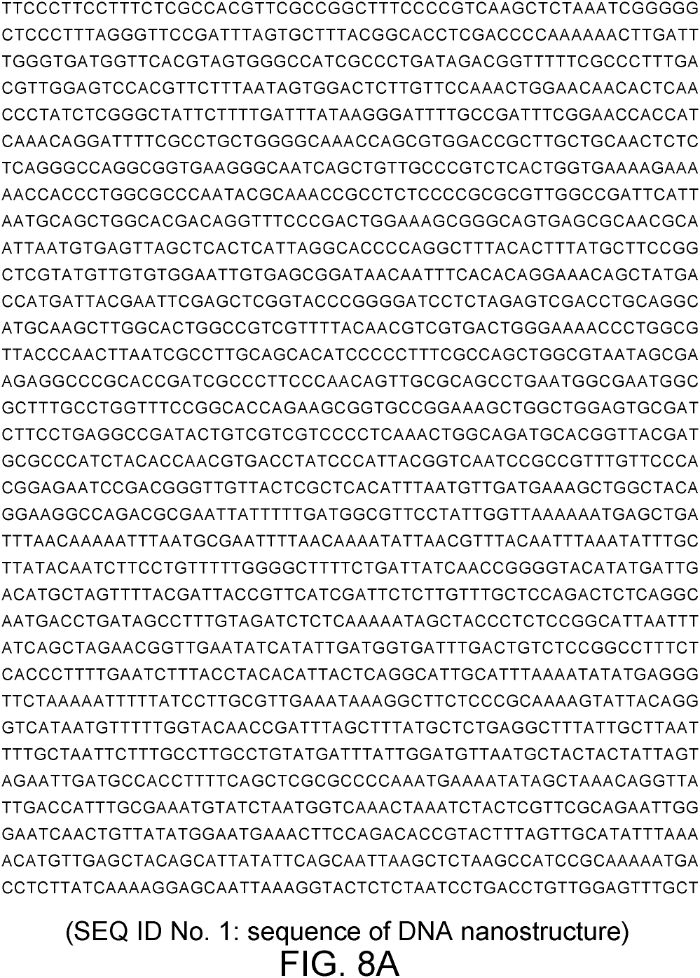

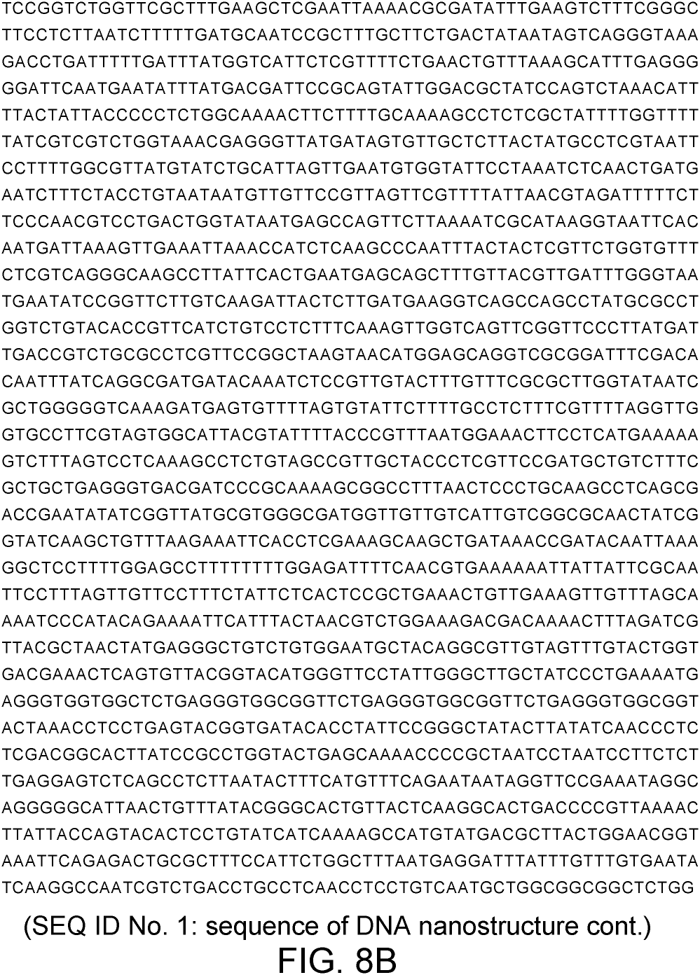

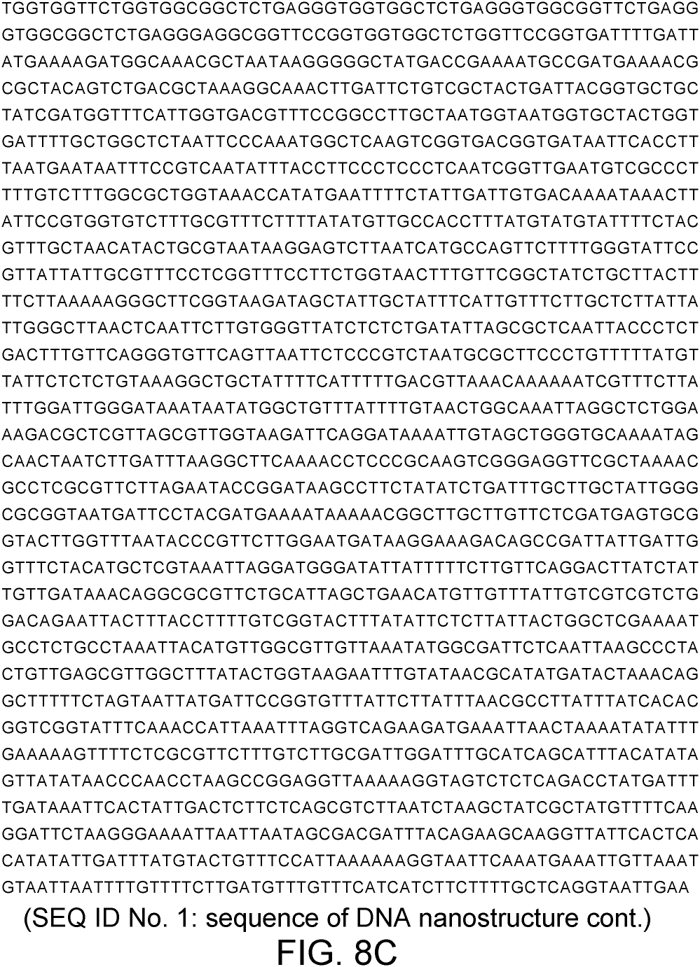

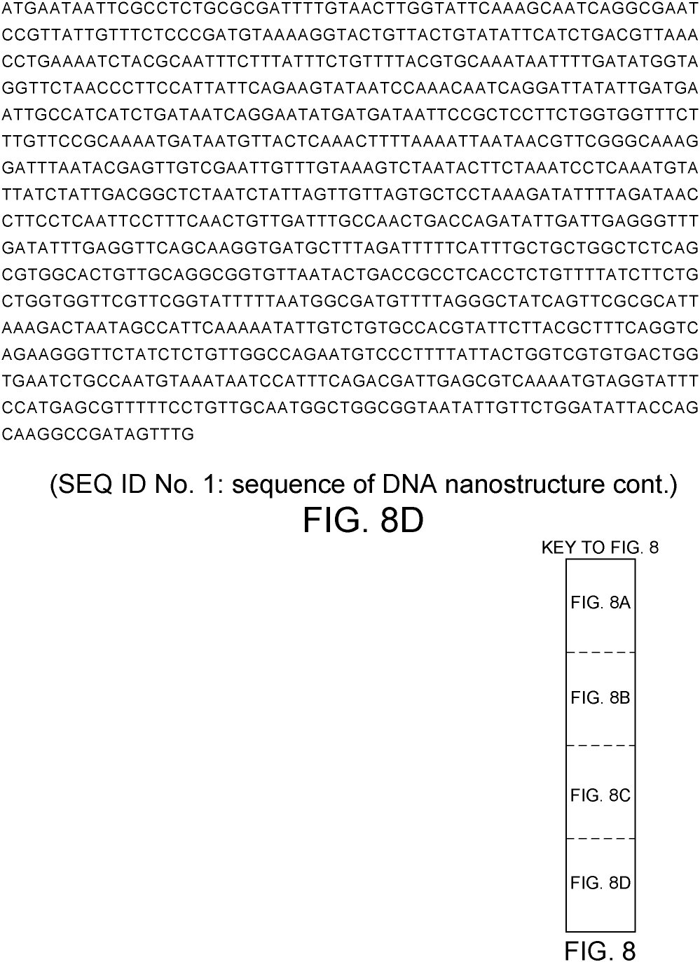

FIGS. 8A, 8B, 8C and 8D list the nucleotide sequence (SEQ ID No: 1) of the circular DNA scaffold of the DNA nanostructure. The circular strand of DNA is taken from the gene of the bacteriophage M13mp18. Because the rail strands that make up DNA nanostructure 40 are formed by a circular strand of DNA, any starting point of the nucleotide sequence is arbitrary. The beginning of the sequence of FIGS. 8A-8D and SEQ ID No: 1 is located on the circular strand of DNA at the bottom of the layout in FIG. 7B. The nucleotide sequence begins: TTCCCTTCCTTT etc. The sequence list (SEQ ID No: 1) of the circular strand of DNA includes 6912 nucleotides, which corresponds to all of the nucleotides shown in the layout of FIGS. 7A, 7B, 7C and 7D. In another embodiment, however, the DNA nanostructure is formed by a circular strand comprising 7249 nucleotides. The DNA nanostructure still has a rectangular shape formed from 6912 nucleotides. However, a loop of 337 (7249-6912) nucleotides of the circular strand protrudes from the rectangular shape. For additional details on making DNA nanostructures, see U.S. Pat. Application Publication 2019/0271647, which is incorporated herein by reference in its entirety.

Returning the steps of method 10, steps 11-16 are repeated in an analogous manner for the second bacterial species, in this example Bacteroides helcogenes.

In first step 17, the second bacterial species of the subset is selected from various bacterial species in the database.

In step 18, a primary binding site is selected on a second rRNA subunit of the second bacterial species.

In step 19, a secondary binding site is selected on the second rRNA subunit of the second bacterial species.

In step 20, second immobilizing binders are formed that include a nucleotide sequence complementary to that of the primary binding site on the second rRNA subunit.

In step 21, second fluorophore binders are formed that include a nucleotide sequence complementary to that of the secondary binding site on the second rRNA subunit.

In step 22, the second fluorophore binders are attached to second DNA nanostructures. The second DNA nanostructures are similar to the first DNA nanostructures except for the combined color that the second DNA nanostructures emit. Each of the second DNA nanostructures is attached to a fourth organic fluorophore having the first color, a fifth organic fluorophore having the second color, and a sixth organic fluorophore having the third color. The intensity by which at least one of the colors is emitted from the second DNA nanostructures must be different than the intensity by which that color is emitted from the first DNA nanostructures so that the combined colors emitted by the two nanostructures are different and the two bacterial species can be distinguished. The intensities by which each of the first, second and third colors fluoresce is controlled by varying the number of individual organic fluorophore molecules that are attached to the DNA nanostructures. Thus, the second DNA nanostructures exhibit a second fluorophore color produced by a second combination of intensities of the first color, the second color and the third color, and the second fluorophore color is distinguishable from the first fluorophore color emitted by the first DNA nanostructures.

In step 23, immobilizing oligonucleotides 45 are attached to a surface 46 of a microscopy chamber, as illustrated in FIG. 3. In one embodiment, each immobilizing oligonucleotide 45 is attached to the glass surface 46 through protein-ligand (biotin-streptavidin) coupling. Biotin bovine serum albumin (bBSA) 47 supplies the biotin that passivates the surface 46 and attaches the streptavidin 48. The surface 46 of the microscopy chamber is thereby coated with streptavidin 48, which is then hybridized to the immobilizing oligonucleotide 45. The immobilizing oligonucleotides 45 also include biotin, which binds to the streptavidin 48. In a second embodiment, the immobilizing oligonucleotides 45 are attached to the surface 46 of the microscopy chamber by being attached directly to the biotin bovine serum albumin (bBSA) 47, and there is no intermediary streptavidin. The second embodiment is illustrated in FIG. 9.

In step 24, the first immobilizing binders 38 are attached to a first group of the immobilizing oligonucleotides 45.

In step 25, the second immobilizing binders are attached to a second group of the immobilizing oligonucleotides 45. In one embodiment, steps 24-25 are performed at the same time. For example, both the first immobilizing binders 38 and the second immobilizing binders are added in the same concentrations to the microscopy chamber so that the immobilizing oligonucleotides 45 are equally and evenly attached to both the first immobilizing binders 38 and the second immobilizing binders. Where more than two species of bacteria are being quantified, the immobilizing binders for each of the bacterial species are equally and evenly attached to the immobilizing oligonucleotides 45. Despite the fact that all of the immobilizing oligonucleotides 45 never are attached to just the immobilizing binder for a particular bacterial species, there will be sufficient immobilizing binders for the rRNA subunits of any particular bacterial species because the concentration of rRNA subunits for that species added later to the microscopy chamber is orders of magnitude lower than the concentration in the chamber of immobilizing binders for the rRNA subunits of the particular species. For example, the rRNA subunits are later added at a concentration in the pico molar (pM) range, whereas the immobilizing binders for the particular bacterial species are present and immobilized in the chamber at a concentration in the micro molar (µM) range.

In step 26, rRNA subunits from bacteria present in a gastrointestinal microbiomic sample from the patient are extracted. The bacteria cells in the sample are destroyed in order to purify the 16S rRNA subunits. In this example, 16S rRNA subunits from both Coraliomargarita akajimensis and Bacteroides helcogenes are extracted, as well as all of the other bacteria strains present in the sample. In one implementation, the microbiomic sample is a stool sample. In other implementations, the microbiomic sample is a sewer sample, a skin swab or a saliva sample.

In step 27, the extracted rRNA subunits are added to the microscopy chamber.

In step 28, hybridization reactions are performed to bind the first immobilizing binders 38 to the primary binding site 36 on the first rRNA subunits 35 of the first bacterial species that are present in the microscopy chamber.

In step 29, hybridization reactions are performed to bind the second immobilizing binders to the primary binding site on the second rRNA subunits of the second bacterial species that are present in the microscopy chamber.

In step 30, hybridization reactions are performed to bind the first fluorophore binders 39 to the secondary binding site 37 on the first rRNA subunits 35 of the first bacterial species that are present in the microscopy chamber.

In step 31, hybridization reactions are performed to bind the second fluorophore binders to the secondary binding site on the second rRNA subunits of the second bacterial species that are present in the microscopy chamber. After the incubation performed in steps 28-31, the unbound rRNA subunits are flushed out of the microscopy chamber. After the chamber has been flushed, the identifiable rRNA subunits of the various bacterial species, for which immobilizing binders and fluorophore binders were synthesized, have been immobilized on the surface of the microscopy chamber.

Steps 30-31 of performing hybridization reactions to bind the first and second fluorophore binders to the secondary binding site on the first and second rRNA subunits, respectively, can be performed before steps 28-29 of binding the first and second immobilizing binders to the primary binding site on the first and second rRNA subunits, respectively. In addition, steps 30-31 of performing hybridization reactions to bind the first and second fluorophore binders to the secondary binding site on the first and second rRNA subunits, respectively, can be performed before the steps 16 and 22 of attaching the first and second fluorophore binders to the first and second DNA nanostructures, respectively.

In step 32, image analysis is performed to detect the first fluorophore color and thereby count each first DNA nanostructure 40 that is immobilized on the surface 46 of the microscopy chamber.

In step 33, image analysis is performed to detect the second fluorophore color and thereby count each second DNA nanostructure that is immobilized on the surface 46 of the microscopy chamber. The microscope used to obtain the images that are analyzed in steps 32-33 can be a Zeiss AxioObserver 7 with 60x/1.4 objective, Colibri 7 illumination and AxioCam 506. In one embodiment, a digital image of the results of the image analysis from steps 32-33 is produced. The image analysis is performed using image processing operations, such as convolution, thresholding, dilution and erosion, and spot identification. Each of the multi-colored spots corresponding to a particular bacterial species is counted on the image of the surface 46 produced in steps 32-33.

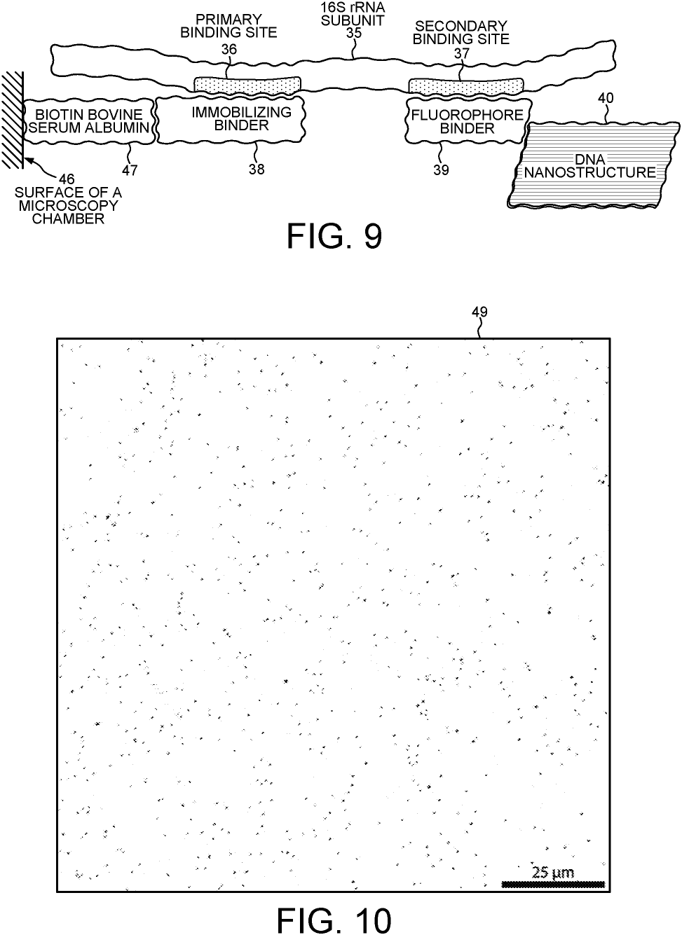

FIG. 10 shows an exemplary digital image 49 generated using the image analysis of steps 32-33. Image 49 is the black/white inverse of the actual image output by the fluorescence microscopy system. Each of the dots in image 49 is a multi-colored spot corresponding to a particular bacterial species. In this example where DNA nanostructures attach only to the first bacterial species and the second bacterial species, there are multi-colored spots with only two combined fluorophore colors. The human eye perceives the two combined fluorophore colors as two distinct colors, whereas the fluorescence microscopy system senses the two combined colors as two distinct combinations of colors.

In step 34, the relative concentration of the first bacterial species compared to the second bacterial species is determined based on how many first DNA nanostructures 40 and how many second DNA nanostructures are counted on the surface 46 of the microscopy chamber. The number of first DNA nanostructures 40 corresponds to the number of first 16S rRNA subunits 35 that were immobilized on the surface 46 of the microscopy chamber, which in turn corresponds to the number of cells of the first bacterial species Coraliomargarita akajimensis in the sample from which the rRNA subunits were extracted in step 26. The number of second DNA nanostructures corresponds to the number of second rRNA subunits that were immobilized on the surface 46 of the microscopy chamber, which in turn corresponds to the number of cells of the second bacterial species Bacteroides helcogenes in the sample from which the rRNA subunits were extracted in step 26.

In the simplified case of applying method 10 to determine the relative concentrations of the first and second species of bacteria listed in FIG. 1, the relative concentration of the first bacterial species compared to the second bacterial species is determined to be 76.8% (21.36/(21.36+6.45)) of the first bacteria Coraliomargarita akajimensis and 23.2% (6.45/(21.36+6.45)) of the second bacteria Bacteroides helcogenes. Thus, the method has determined that there is an overabundance of the first bacterial species Coraliomargarita akajimensis and an under-abundance of the second bacterial species Bacteroides helcogenes.

In a subsequent step of method 10, an optimal relative concentration of the first bacterial species compared to the second bacterial species is determined that will improve a medical condition of the patient from whom the gastrointestinal microbiomic sample was taken. Then a dietary supplement is administered to the patient that changes the relative concentration of the first bacterial species compared to the second bacterial species so as to move the patient closer to having the optimal relative concentration of the two bacterial species.

In another example, a therapy is administered in order to improve a medical condition that is correlated to the quantitative composition of the gastrointestinal microbiome of the patient. In one implementation of method 10, the comparing of the quantitative microbiomic fingerprint of the patient with the reference data of diabetic patients indicates that the patient will develop diabetes. Thus, a medical therapy for treating diabetes is administered to the patient, such as administering insulin. Two of the possible bacteria of this quantitative fingerprint are Clostridiales and Sutterella genera. In yet another example, the quantitative fingerprint correlates to high blood pressure, and an antihypertensive medication is administered to the patient. In yet another example, the quantitative fingerprint indicates an increased risk for atherosclerosis, and a treatment to slow the progression of atherosclerosis is initiated, such as administering statin drugs. The quantitative fingerprint indicative of atherosclerosis includes bacteria species that are involved in the metabolism of cholesterol and lipids.

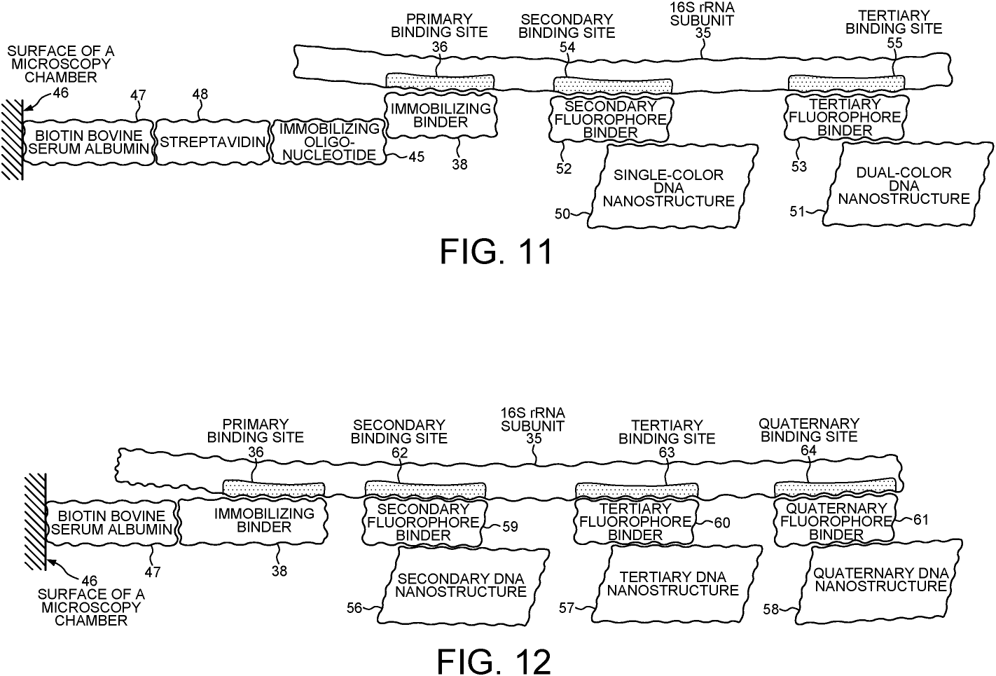

FIG. 11 illustrates another embodiment in which each target bacterial species is identified and quantified by being attached to two separate fluorophore binders and thereby to two fluorescent DNA nanostructures. In the embodiment of FIG. 11, one of the three colors that together in combination produce the fluorophore color that identifies a particular bacterial species is emitted from a DNA nanostructure 50 separate from the DNA nanostructure 51 from which the other two colors are emitted. Thus, two separate DNA nanostructures 50-51 are used to produce the three colors which in combination result in the fluorophore color associated with the 16S rRNA subunit of the particular target bacterial species. In this embodiment, a binding site is selected for each of the two separate DNA nanostructures 50-51, which are attached via corresponding fluorophore binders 52-53, respectively.

After a primary binding site is selected on the rRNA subunit of the target bacterial species in order to attach the immobilizing binder 38, a secondary binding site 54 and a tertiary binding site 55 are selected. The secondary fluorophore binders 52 include a nucleotide sequence that is complementary to that of the secondary binding site 54 on the rRNA subunit 35 of the target bacterial species. And the tertiary fluorophore binders 53 include a nucleotide sequence that is complementary to that of the tertiary binding site 55 on the rRNA subunit 35. In this manner, each immobilized rRNA subunit 35 of the target bacterial species becomes attached to both a single-color DNA nanostructure 50 and a dual-color DNA nanostructure. Single-color DNA nanostructure 50 is attached to first organic fluorophores that fluoresce the first color. Dual-color DNA nanostructure 51 is attached both to second organic fluorophores that fluoresce the second color and to third organic fluorophores that fluoresce the third color. For example, the first organic fluorophore is Atto 647N and fluoresces red, the second organic fluorophore is Cy3 and fluoresces green, and the third organic fluorophore is Atto 488 and fluoresces blue. The fluorophore color that identifies the target bacterial species is produced by the combination of the intensity of the first color emitted by the predetermined number of first organic fluorophores on DNA nanostructure 50 and the intensities of the second and third colors emitted by the predetermined number of second and third organic fluorophores on DNA nanostructure 51. Thus, the various intensities of the three colors of light emitted by the three types of fluorophores combine and are sensed by a fluorescence microscopy system as a single multi-colored spot that identifies the target bacterial species.

FIG. 12 illustrates yet another embodiment in which each target bacterial species is identified and quantified by being attached to three separate fluorophore binders and thereby to three fluorescent DNA nanostructures. In the embodiment of FIG. 12, each of the three colors that together in combination produce the fluorophore color that identifies a particular bacterial species is emitted from a separate DNA nanostructure. Thus, three separate DNA nanostructures 56-58 are used to produce the three colors which in combination result in the fluorophore color associated with the 16S rRNA subunit of the particular target bacterial species. A separate binding site is selected for each of the three DNA nanostructures 56-58, which are attached via corresponding fluorophore binders 59-61, respectively.

After a primary binding site is selected on the rRNA subunit of the target bacterial species in order to attach the immobilizing binder 38, a secondary binding site 59, a tertiary binding site 60 and a quaternary binding site 61 are selected. The secondary fluorophore binders 59 include a nucleotide sequence that is complementary to that of the secondary binding site 62 on the rRNA subunit 35 of the target bacterial species. The tertiary fluorophore binders 60 include a nucleotide sequence that is complementary to that of the tertiary binding site 63. And the quaternary fluorophore binders 60 include a nucleotide sequence that is complementary to that of the tertiary binding site 63.

In this manner, each immobilized rRNA subunit 35 of the target bacterial species becomes attached to three single-color DNA nanostructures 56-58. DNA nanostructure 56 is attached to first organic fluorophores that fluoresce the first color. DNA nanostructure 57 is attached to second organic fluorophores that fluoresce the second color. And DNA nanostructure 58 is attached to third organic fluorophores that fluoresce the third color. In one implementation, the first organic fluorophore is Atto 647N and fluoresces red, the second organic fluorophore is Cy3 and fluoresces green, and the third organic fluorophore is Atto 488 and fluoresces blue. The fluorophore color that identifies the target bacterial species is produced by the combination of the intensity of the first color emitted by a predetermined number of first organic fluorophores on DNA nanostructure 56, the intensity of the second color emitted by a predetermined number of second organic fluorophores on DNA nanostructure 57, and the intensity of the third color emitted by a predetermined number of third organic fluorophores on DNA nanostructure 58. The various intensities of the three colors of light emitted by the three types of fluorophores on the three DNA nanostructures combine and are sensed by a fluorescence microscopy system as a single multi-colored spot that identifies the target bacterial species.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

1. A method comprising:
selecting a first bacterial species;
selecting a primary binding site on a first ribosomal RNA (rRNA) subunit of the first bacterial species;
selecting a secondary binding site on the first rRNA subunit;
forming first immobilizing binders comprising a nucleotide sequence complementary to that of the primary binding site on the first rRNA subunit;
forming first fluorophore binders comprising a nucleotide sequence complementary to that of the secondary binding site on the first rRNA subunit;
attaching the first fluorophore binders to first DNA nanostructures each comprising parallel DNA double helices that form a flat shape, wherein the first DNA nanostructures have maximum lengths of less than 150 nm, wherein each of the first DNA nanostructures is attached to a first organic fluorophore with a first color, a second organic fluorophore with a second color, and a third organic fluorophore with a third color, and wherein the first DNA nanostructures exhibit a first fluorophore color produced by a first combination of intensities of the first color, the second color and the third color;
selecting a second bacterial species;
selecting a primary binding site on a second rRNA subunit of the second bacterial species;
selecting a secondary binding site on the second rRNA subunit;
forming second immobilizing binders comprising a nucleotide sequence complementary to that of the primary binding site on the second rRNA subunit;
forming second fluorophore binders comprising a nucleotide sequence complementary to that of the secondary binding site on the second rRNA subunit;
attaching the second fluorophore binders to second DNA nanostructures each comprising parallel DNA double helices, wherein the second DNA nanostructures have maximum lengths of less than 150 nm, wherein each of the second DNA nanostructures is attached to a fourth organic fluorophore with the first color, a fifth organic fluorophore with the second color, and a sixth organic fluorophore with the third color, and wherein the second DNA nanostructures exhibit a second fluorophore color produced by a second combination of intensities of the first color, the second color and the third color;
attaching immobilizing oligonucleotides to a surface of a microscopy chamber;
attaching the first immobilizing binders to a first group of the immobilizing oligonucleotides;
attaching the second immobilizing binders to a second group of the immobilizing oligonucleotides;
extracting rRNA subunits from bacteria present in a gastrointestinal microbiomic sample;
adding the extracted rRNA subunits to the microscopy chamber;
performing hybridization reactions to bind the first immobilizing binders to the primary binding site on first rRNA subunits present in the microscopy chamber;
performing hybridization reactions to bind the second immobilizing binders to the primary binding site on second rRNA subunits present in the microscopy chamber;
performing hybridization reactions to bind the first fluorophore binders to the secondary binding site on first rRNA subunits present in the microscopy chamber;
performing hybridization reactions to bind the second fluorophore binders to the secondary binding site on second rRNA subunits present in the microscopy chamber;
performing image analysis to detect the first fluorophore color and thereby count each first DNA nanostructure that is immobilized on the surface of the microscopy chamber;
performing image analysis to detect the second fluorophore color and thereby count each second DNA nanostructure that is immobilized on the surface of the microscopy chamber; and
determining a relative concentration of the first bacterial species compared to the second bacterial species based on how many first DNA nanostructures and how many second DNA nanostructures are counted on the surface of the microscopy chamber.

2. The method of claim 1, further comprising:
determining an optimal relative concentration of the first bacterial species compared to the second bacterial species to improve a medical condition of a patient from whom the gastrointestinal microbiomic sample was taken; and
administering a dietary supplement to the patient that changes the relative concentration of the first bacterial species compared to the second bacterial species so as to move closer to the optimal relative concentration.

3. The method of claim 1, wherein no first organic fluorophore is attached to any of the first DNA nanostructures at a location within 5 nm of any second organic fluorophore or third organic fluorophore.

4. The method of claim 1, wherein the first fluorophore color is one of 64 possible colors produced by 64 combinations of four intensities of the first color, the second color and the third color being emitted from fluorescent sites on each of the first DNA nanostructures.

5. The method of claim 1, wherein the first fluorophore color is one of 8 possible colors produced by 8 combinations of two intensities of the first color, the second color and the third color being emitted from fluorescent sites on each of the first DNA nanostructures.

6. The method of claim 1, wherein the flat shape is a rectangle, and wherein the rectangle has dimensions of 50 nm-70 nm by 80 nm-100 nm.

7. The method of claim 1, wherein the first DNA nanostructures have maximum lengths of less than 110 nm.

8. The method of claim 1, wherein the step of performing hybridization reactions to bind the first fluorophore binders to the secondary binding site on the first rRNA subunits is performed before the step of attaching the first fluorophore binders to the first DNA nanostructures.

9. The method of claim 1, wherein the first color is red and the first organic fluorophore is Atto 647N, wherein the second color is green and the second organic fluorophore is Cy3, and wherein the third color is blue and the third organic fluorophore is Atto 488.

10. The method of claim 1, wherein the second organic fluorophore has a wavelength of maximum absorption that is at least 25 nm larger than a wavelength of maximum emission of the third organic fluorophore, and wherein the first organic fluorophore has a wavelength of maximum absorption that is at least 25 nm larger than a wavelength of maximum emission of the second organic fluorophore.

11. The method of claim 1, wherein the first rRNA subunit is selected from the group consisting of: a 16S ribosomal RNA subunit, an 18S ribosomal RNA subunit, a 23S ribosomal RNA subunit, and a 28S ribosomal RNA subunit.

12. The method of claim 1, wherein the first rRNA subunit is comprised of more than 1000 nucleotides and less than 5000 nucleotides.

13. The method of claim 1, wherein the primary binding site on the first rRNA subunit includes more than 18 nucleotides and less than 26 nucleotides.

14. The method of claim 1, wherein the primary binding site on the first rRNA subunit includes at least 20 nucleotides separated into a first sequence having at least 10 nucleotides and a second sequence having at least 10 nucleotides, and wherein the nucleotide sequence of the immobilizing binders includes a first region that is complementary to the first sequence, a second region that is complementary to the second sequence, and a third region that is not complementary to the primary binding site on the first rRNA subunit.

15. The method of claim 1, wherein the primary binding site on the first rRNA subunit includes a sequence of no more than four repetitive nucleotides.

16. The method of claim 1, wherein the primary binding site on the first rRNA subunit includes nucleotides comprising 35%-55% Guanine and Cytosine.

17. The method of claim 1, wherein the surface of the microscopy chamber is coated with streptavidin, wherein the immobilizing oligonucleotides include biotin, and wherein the biotin binds to the streptavidin.

18. A method comprising:
selecting a primary binding site and a secondary binding site on a first ribosomal RNA (rRNA) subunit of a first bacterial species;
forming first immobilizing binders comprising a nucleotide sequence complementary to that of the primary binding site on the first rRNA subunit;
forming first fluorophore binders comprising a nucleotide sequence complementary to that of the secondary binding site on the first rRNA subunit;
attaching the first fluorophore binders to first DNA nanostructures each comprising parallel DNA double helices that form a flat shape, wherein the first DNA nanostructures have maximum dimensions of less than 300 nm, wherein each of the first DNA nanostructures is attached to a first organic fluorophore with a first color and a second organic fluorophore with a second color, and wherein the first DNA nanostructures exhibit a first fluorophore color produced by a first combination of intensities of the first color and the second color;
selecting a primary binding site and a secondary binding site on a second rRNA subunit of a second bacterial species;
forming second immobilizing binders comprising a nucleotide sequence complementary to that of the primary binding site on the second rRNA subunit;
forming second fluorophore binders comprising a nucleotide sequence complementary to that of the secondary binding site on the second rRNA subunit;
attaching the second fluorophore binders to second DNA nanostructures each comprising parallel DNA double helices, wherein each of the second DNA nanostructures is attached to a third organic fluorophore with the first color and a fourth organic fluorophore with the second color, and wherein the second DNA nanostructures exhibit a second fluorophore color produced by a second combination of intensities of the first color and the second color;
attaching the first immobilizing binders to a surface of a microscopy chamber;
attaching the second immobilizing binders to the surface of a microscopy chamber;
extracting rRNA subunits from bacteria present in a gastrointestinal microbiomic sample of a patient;
adding the extracted rRNA subunits to the microscopy chamber;
performing hybridization reactions to bind the first immobilizing binders to the primary binding site on first rRNA subunits present in the microscopy chamber;
performing hybridization reactions to bind the second immobilizing binders to the primary binding site on second rRNA subunits present in the microscopy chamber;
performing hybridization reactions to bind the first fluorophore binders to the secondary binding site on first rRNA subunits present in the microscopy chamber;
performing hybridization reactions to bind the second fluorophore binders to the secondary binding site on second rRNA subunits present in the microscopy chamber;
performing image analysis to detect the first fluorophore color and thereby count each first DNA nanostructure that is immobilized on the surface of the microscopy chamber;
performing image analysis to detect the second fluorophore color and thereby count each second DNA nanostructure that is immobilized on the surface of the microscopy chamber; and
determining a relative concentration of the first bacterial species compared to the second bacterial species based on how many first DNA nanostructures and how many second DNA nanostructures are counted on the surface of the microscopy chamber.

19. The method of claim 18, further comprising:
determining an optimal relative concentration of the first bacterial species compared to the second bacterial species to improve a medical condition of the patient; and
administering a dietary supplement to the patient that changes the relative concentration of the first bacterial species compared to the second bacterial species so as to move closer to the optimal relative concentration.

20. The method of claim 18, wherein the flat shape is a rectangle, and wherein the rectangle has dimensions of 50 nm-70 nm by 80 nm-100 nm.

21. The method of claim 18, wherein the step of attaching the first immobilizing binders to the surface of the microscopy chamber is performed before the step of attaching the first fluorophore binders to the first DNA nanostructures.

22-23. (canceled)
